(19) United States
(12) Patent Application Publication
(10) Pub. No.: US 2025/0197361 A1
(43) Pub. Date: Jun. 19, 2025

(54) Trimerization catalysts

(71) Applicant: Evonik Operations GmbH, Essen (DE)

(72) Inventors: Emmanouil SPYROU, Schermbeck (DE); Elke GOLLAN, Herne (DE); Christine APPEL, Recklinghausen (DE); Ralf KLAWIKOWSKI, Recklinghausen (DE); Jennifer EGGERT, Gelsenkirchen (DE); Stephan KOHLSTRUK, Gladbeck (DE)

(73) Assignee: Evonik Operations GmbH, Essen (DE)

(21) Appl. No.: 18/982,021

(22) Filed: Dec. 16, 2024

(30) Foreign Application Priority Data
Dec. 14, 2023 (EP) 23216564.7

(51) Int. Cl. C07D 251/30; B01J 37/04; C07C 269/02

(52) U.S. Cl. CPC C07D 251/30; B01J 37/04; C07C 269/02; C07C 2531/02

(57) ABSTRACT

The present invention relates to ammonium salts of formula (I)

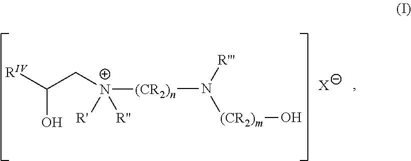

where R=—H, —CH₃, —CH₂CH₃; n=1, 2, 3, 4, 5; m=2, 3; R′, R″, R′″=—CH₃, —CH₂CH₃; R^{IV}=—H, —CH₃, —CH₂CH₃ and X=monovalent counterion, to a process for their production and to the use thereof.

The present invention relates to novel ammonium salts employable as catalysts, especially as catalysts for reactions with isocyanate-containing compounds. The present invention further relates to a process for their production and to the use thereof.

Reactions of isocyanate-containing compounds are of great interest for industrial chemistry. Especially the trimerization of isocyanates to isocyanurates and the conversion of isocyanates and hydroxy-containing compounds to urethane-containing compounds are of great industrial importance, since the corresponding products are used in many fields of application.

Polyisocyanurates are valuable input materials for the production of high-quality coatings with good mechanical properties and good resistance to light and weathering. Polyisocyanurates based on isophorone diisocyanate (IPDI) are also used as raw materials for PUR-based elastomer applications.

Polyisocyanurates are in principle obtained by catalytic trimerization of suitable isocyanates. Trimerizable isocyanates include for example aromatic, cycloaliphatic and aliphatic difunctional and higher-functional polyisocyanates. Contemplated catalysts include for example tertiary amines (DE 24 52 532 A1), complexes of basic alkali metal compounds and acyclic organic compounds (EP 0 056 159 A1), aminosilyl-containing compounds (U.S. Pat. No. 4,697,014) and quaternary ammonium salts (U.S. Pat. No. 4,503,226).

Catalysts particularly suitable for trimerization of isocyanates to isocyanurates further include quaternary hydroxyalkylammonium salts.

DE 26 31 733 A1 for example discloses the catalysis of trimerizations and urethane syntheses with quaternary hydroxyalkylammonium salts having a beta-hydroxyalkyl group.

DE 29 16 201 A1 also teaches that quaternary N-(hydroxyalkyl) ammonium salts can be used for trimerization of isocyanates to isocyanurates.

EP 1 454 933 A1 teaches that trimerization can employ quaternary beta-hydroxylated ammonium salts whose quaternary nitrogen atom bears three radicals X which together form a tricyclic ring. The tricyclic ring is formed via a further common nitrogen atom which may be hydroxyalkylated (and is then accordingly also in quaternary form).

Finally, DE 41 15 402 A1 discloses in the examples the production of N-(hydroxyalkyl) ammonium salts which may be used for trimerization of isocyanurates. It is always the case, inter alia, that one equivalent of N, N,N′-trimethyl-N′-(β-hydroxyethyl)-ethylenediamine is reacted with two equivalents of monocarboxylic acid before an excess of a monoepoxide is added, such that the resulting ammonium salt comprises two quaternary nitrogen atoms.

The ammonium salts disclosed in the prior art already solve many of the problems of the prior art, for example that the isocyanurates producible with them are storage-stable, low-colour and low-odour. However, the disadvantage is that the known catalysts are not yet sufficiently active.

The problem addressed by the present invention is accordingly that of providing catalysts which, similarly to the known ammonium salts, make it possible to produce storage-stable, low-colour and low-odour isocyanurates but which have the advantage of greater activity.

This problem is solved by the ammonium salt according to the invention having the following formula (I)

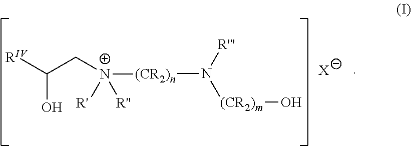

where
R=—H, —CH₃, —CH₂CH₃
n=1, 2, 3, 4, 5,
m=2, 3,
R′, R″, R′″=—CH₃, —CH₂CH₃
R^{IV}=H, —CH₃, —CH₂CH₃
and X=a monovalent counterion.

The radicals R may independently of one another be selected from the group consisting of —H, —CH₃, —CH₂CH₃.

The ammonium salts according to the invention are ammonium salts producible by reaction of a ditertiary amine, where one of the tertiary amino groups is substituted with a hydroxyalkyl group, with an alkylene oxide and an acid HX. If the alkylene oxide and the acid are not present in a molar excess based on the total amount of the ditertiary amine, surprisingly only the amino group without the hydroxyalkyl group —(CR₂)ₘ—OH reacts with the alkylene oxide. Corresponding salts having an ammonium and a tertiary amino group in the cation are surprisingly more active than diammonium salts.

Preferred catalyst properties result when the ammonium salts are derived from acids HX selected from the group of carboxylic acids. It is further preferable when the carboxylic acids are selected from the group of aliphatic and heterocyclic carboxylic acids. It is yet more preferable when the carboxylic acid is an aliphatic carboxylic acid. It is yet more preferable when the carboxylic acid is an alkanoic acid or alkenoic acid. Particularly preferred carboxylic acids may be selected from the group of alkanoic acids having the formula R^{V}COOH where R^{V}=CᵣH₂ᵣ₊₁ where r=1-9. Corresponding ammonium salts have the formula (II):

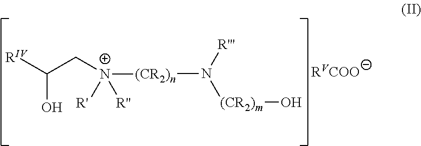

In the formulae (I) and (II) the radicals R are independently of one another selected from the group consisting of —H, —CH₃ and —CH₂CH₃. It is preferable when R is independently at each occurrence selected from the group consisting of —H and —CH₃. It is more preferable when R is independently at each occurrence selected from the group consisting of —H and —CH₃ with the proviso that each carbon atom bears at most one radical R that is a —CH₃ radical. It is yet more preferable when all R=—H.

In formulae (I) and (II) the index n is a number selected from 1, 2, 3, 4 and 5. It is preferable when n is a number selected from 2, 3 and 4. It is it yet more preferable when n=2 or 3. It is very particularly preferable when n=2.

In formulae (I) and (II) the index m is a number selected from 2 and 3. It is yet more preferable when m=2.

The radicals R′, R″ and R″ may independently of one another be selected from the group consisting of —CH₃ and —CH₂CH₃. It is preferable when each of the radicals R′, R″ and R′″=—CH₃.

The radical R^{IV} in formulae (I) and (II), which is derived from the alkylene oxide used to produce the ammonium salt, is selected from the group consisting of —H, —CH₃ and —CH₂CH₃. Accordingly the radical R^{IV} is derived from an alkylene oxide selected from ethylene oxide, propylene oxide and α-butylene oxide. It is preferable when the radical R^{IV} is selected from the group consisting of —CH₃ and —CH₂CH₃. Due to the combination of comparatively low toxicity and simple handling (above all due to the relatively high boiling point) of the α-butylene oxide necessary for the corresponding synthesis, it is very particularly preferable when R^{IV} is —CH₂CH₃.

The present invention further relates to a process for producing the ammonium salts of formula (II) according to the invention in which a ditertiary amine, where one of the tertiary amino groups is substituted with a hydroxyalkyl group, is reacted with an alkylene oxide and an acid HX, preferably an alkanoic acid, in particular an alkanoic acid having the formula R^{V}COOH where R^{V}=CᵣH₂ᵣ₊₁ where r=1-9.

The alkylene oxide is preferably employed in a molar ratio of 0.9-1.1, more preferably 0.95-1.05, yet more preferably 0.98-1.02, and the acid in a molar ratio of 0.9-1.1, more preferably 0.95-1.05, yet more preferably 0.98-1.02, in each case based on the molar amount of the ditertiary amine.

The ditertiary amine preferably has the following formula (III)

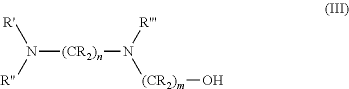

where
R=—H, —CH₃, —CH₂CH₃
n=1, 2, 3, 4, 5,
m=2, 3 and
R′, R″, R′″=—CH₃, —CH₂CH₃.

The radical R is independently at each occurrence selected from the group consisting of —H, —CH₃ and —CH₂CH₃. It is preferable when R is independently at each occurrence selected from the group consisting of —H and —CH₃. It is more preferable when R is independently at each occurrence selected from the group consisting of —H and —CH₃ with the proviso that each carbon atom bears at most one radical R that is a —CH₃ radical. It is yet more preferable when all R=—H.

The index n is a number selected from 1, 2, 3, 4 and 5. It is preferable when n is a number selected from 2, 3 and 4. It is it yet more preferable when n=2 or 3. It is very particularly preferable when n=2.

The index m is a number selected from 2 and 3. It is yet more preferable when m=2.

The radicals R′, R″ and R″ may independently of one another be selected from the group consisting of —CH₃ and —CH₂CH₃. It is preferable when each of the radicals R′, R″ and R′″=—CH₃.

It is preferable to employ acids HX selected from the group of carboxylic acids. It is further preferable when the carboxylic acids are selected from the group of aliphatic and heterocyclic carboxylic acids. It is yet more preferable when the carboxylic acid is an aliphatic carboxylic acid. It is yet more preferable when the carboxylic acid is an alkanoic acid or alkenoic acid. Particularly preferred carboxylic acids may be selected from the group of alkanoic acids. Particularly preferred alkanoic acids may be selected from the group of alkanoic acids having the formula R^{V}COOH where R^{V}=CᵣH₂ᵣ₊₁ where r=1-9.

The alkylene oxide is yet more preferably selected from ethylene oxide, propylene oxide and α-butylene oxide. Corresponding products conform to formula (III). The alkylene oxide is yet more preferably selected from propylene oxide and α-butylene oxide. Due to the combination of comparatively low toxicity and simple handling (above all due to the relatively high boiling point), it is very particularly preferable when the alkylene oxide is α-butylene oxide.

The process according to the invention is preferably performed in such a way that the ditertiary amine is first mixed with the acid and subsequently the alkylene oxide is added.

The process according to the invention may in principle be carried out in the presence or absence of a solvent. However, it is preferably performed in the absence of a solvent. Preferred reaction temperatures are between RT and 120° C., more preferably between 40° C. and 80° C. The reaction may be checked for completeness by gas chromatography for example. As soon as alkylene oxide is no longer detectable the reaction is terminated.

The present invention further provides for the use of the ammonium salts according to the invention as catalysts. Also preferred is the use of the ammonium salts according to the invention as catalysts for trimerization of isocyanates to isocyanurates or for conversion of isocyanates and hydroxy-containing compounds to urethane-containing compounds. The ammonium salts according to the invention are very particularly suitable as catalysts for trimerization of isocyanates to isocyanurates.

### EXAMPLES

### Inventive Example A

146 parts of N,N,N′-trimethyl-N′-(hydroxyethyl)-ethylenediamine (1 mol of diamine) are mixed with 116 parts (1 mol) of hexanoic acid and then admixed portionwise with 72 parts (1 mol) of 1,2-butylene oxide at 40° C. under reflux and stirring. After complete addition the mixture is stirred at 40° C. for a further 96 h after which 1,2-butylene oxide is no longer detectable in the GC. According to NMR only the dimethylamine nitrogen reacted.

### Noninventive Example B (According to DE4115102)

146 parts of N,N,N′-trimethyl-N′-(hydroxyethyl)-ethylenediamine (1 mol of diamine) are mixed with 232 parts (2 mol) of hexanoic acid and then admixed portionwise with 144 parts (2 mol) of 1,2-butylene oxide at 40° C. under reflux and stirring. After complete addition the mixture is stirred at 40° C. for a further 96 h after which 1,2-butylene oxide is no longer detectable in the GC.

### Trimerization

### General Procedure:

1500 g of isophorone diisocyanate (IPDI) are heated to 70° C. and only then mixed with 3 g of catalyst before stirring. The duration of exothermicity, the maximum of the temperature curve and the final NCO number are determined.

| | Duration of | Maximum of | NCO number |
|---|---|---|---|
| | exothermicity | temperature | after reaction |
| | [min] | curve [° C.] | (start 37.8) |
| example A | 2:30 | 156 | 24.5 |
| example B | 2:43 | 137 | 28.6 |

The resulting partial trimerizates are not only stable and colourless but also low-odour. The catalyst according to the invention having one ammonium and one tertiary amino group in the cationic proportion is markedly more active than the catalyst of the comparative example with two ammonium groups in the cationic proportion.

1. Ammonium salt of formula (I) 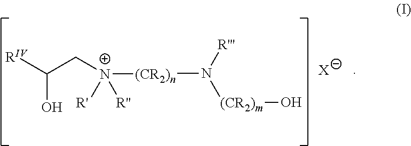
where
R=—H, —CH₃, —CH₂CH₃
n=1, 2, 3, 4, 5
m=2, 3,
R′, R″, R″=—CH₃, —CH₂CH₃
R^{IV}=—H, —CH₃, —CH₂CH₃
and X=a monovalent counterion.

2. Ammonium salt according to claim 1,
wherein the ammonium salt comprises the formula (II) 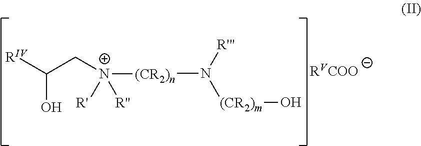
where R^{V}=CᵣH₂ᵣ₊₁ and r=1-9.

3. Ammonium salt according to claim 1, wherein
R=—H.

4. Ammonium salt according to claim 1, wherein
n=2 or 3.

5. Ammonium salt according to claim 1, wherein
m=2.

6. Ammonium salt according to claim 1, wherein
R′=R″=R′″=—CH₃.

7. Ammonium salt according to claim 1, wherein
R^{IV}=—CH₂CH₃.

8. Process for producing an ammonium salt according to claim 1,
wherein
a ditertiary amine, where one of the tertiary amino groups is substituted with a hydroxyalkyl group, is reacted with an alkylene oxide and an acid HX.

9. Process according to claim 8,
wherein
the alkylene oxide is employed in a molar ratio of 0.9-1.1 and the acid in a molar ratio of 0.9-1.1, in each case based on the molar amount of the ditertiary amine.

10. Process according to claim 8,
wherein
the ditertiary amine has the formula 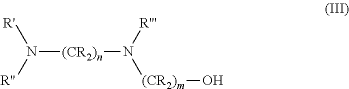
where
R=—H, —CH₃, —CH₂CH₃
n=1, 2, 3, 4, 5,
m=2, 3 and
R′, R″, R″=—CH₃, —CH₂CH₃.

11. Process according to claim 10,
wherein
the acid HX is an alkanoic acid selected from the group of alkanoic acids having the formula R^{V}COOH where R^{V}=CᵣH₂ᵣ₊₁ where r=1-9.

12. Process according to claim 11,
wherein
the alkylene oxide is selected from ethylene oxide, propylene oxide and α-butylene oxide.

13. A method for trimerization of isocyanates to isocyanurates or for conversion of isocyanates and hydroxy-containing compounds to urethane-containing compounds comprising applying the ammonium salt of claim 1 as a catalyst to the isocyanates or the hydroxy-containing compound.
